(19) United States
(12) Patent Application Publication
(10) Pub. No.: US 2007/0255127 A1
(43) Pub. Date: Nov. 1, 2007

(54) Mobile electroencephalograph data collection and diagnosis system

(76) Inventors: Frederick Mintz, Chatsworth, CA (US); Philip I. Moynihan, La Canada, CA (US)

Correspondence Address: TOPE-MCKAY & ASSOCIATES, 23852 PACIFIC COAST HIGHWAY #311, MALIBU, CA, 90265, US

(21) Appl. No.: 11/726,403

(22) Filed: Mar. 20, 2007

Related U.S. Application Data
(60) Provisional application No. 60/783,938, filed on Mar. 20, 2006.

(51) Int. Cl. A61B 5/0408

(52) U.S. Cl. 600/383

(57) ABSTRACT

Described is an electroencephalograph (EEG) data collection system. The system includes a helmet with a plurality of data collection electrodes. Each data collection electrode has a housing and a pressurized probe affixed with the housing. The pressurized probe includes an electrically conductive base for electrical communication with the scalp of a user for detecting EEG signals of the user. The pressurized probe is pressurized such that the electrically conductive base is forced away from the housing and toward a user's scalp. The data collection electrode is further formed to hold a discrete amount of an electrically conductive gel therein and dispense the gel proximate the electrically conductive base to facilitate an electrical communication between the user's scalp and the electrically conductive base. The system also includes Relational Data Base Management System that allows for in vivo EEG data collection, analysis, and diagnosis.

### PRIORITY CLAIM

The present application is a non-provisional patent application, claiming the benefit of priority to U.S. Provisional Application No. 60/783,938, filed on Mar. 20, 2006, entitled, “Mobile in vivo EEG data collection and diagnoses comparison system.”

### BACKGROUND OF THE INVENTION

(1) Field of Invention

The present invention relates to a system for mobile electroencephalographic (EEG) data recording and, more specifically, to a system utilizing electrodes that are capable of both automatic dispensation of an electrically conductive gel and the collection of the EEG data, with a subsystem further being capable of collaborating and analyzing the acquired data.

(2) Description of Related Art

Electroencephalograph (EEG) recording devices have long been known in the art. Since the late 1800's, neural activity has been recorded using EEG probes. Since its discovery more than a century ago, EEG has been become a common tool of the neurologist and the neurosurgeon. EEG is most commonly thought of as a tool used to localize the foci of epileptic seizures in epileptics, the general locations of brain tumors, and regions damaged by stroke.

More recently, smaller EEG systems have been developed that allow free movement of the subject. Such technology has been referred to as both mobile EEG and ambulatory EEG (aEEG). Mobile EEG systems allow subjects to engage in more “day to day” activities than would be permissible if they were attached to non-mobile EEG systems.

In order to enable the EEG system to receive the requisite electrical currents, users typically apply an electrically conductive gel to the user's scalp. The EEG systems are then applied over the gel and attached to the user's scalp. Using the mobile systems described above, a user can apply the gel, the EEG system, and thereafter resume daily activities. However, a problem with such systems is that upon usage and activity, the gel is quickly forced away from the EEG probe. To alleviate such a problem, a few prior art references were devised that dispense an electrically conductive gel.

Examples of such gel-dispensing EEG systems can be found as issued two patents. For example, U.S. Pat. No. 4,709,702 discloses an electroencephalographic cap that has spring loaded electrodes and includes the ability to deliver an electrically conductive solution to each electrode site. The delivery of the electrically conductive solution is accomplished by the use of a hand driven pump that is attached to the head-band of the device. This is undesirable in that it requires the attachment of a pump whenever additional electrically conductive gel is needed. Furthermore, it will deliver additional electrically conductive solution to all sites rather than the specific sites that may have lost their gel to local movements (e.g., chewing or talking will cause portions of the scalp to move more than others).

Second, U.S. Pat. No. 6,640,122 discloses an electroencephalographic recording device that will contain and deliver electrically conductive gel in an automatic fashion via a sponge at the interface of an electrode with the scalp. This method of dispensation will deliver the electrically conductive solution to the most physically active regions of the scalp, however, the use of a sponge as a dispensation mechanism is limiting. For example, a sponge will not be able to maintain electrical contact with a scalp that is covered in hair.

Thus, a continuing need exists for a mobile EEG recording system that automatically delivers electrically conductive gel to the electrode sites and utilizes electrodes that will maintain electrical contact with the scalp through the hair.

### SUMMARY OF INVENTION

The present invention is a mobile electroencephalograph (EEG) data collection and diagnosis system. The system comprises a data collection electrode that has a housing and a pressurized probe affixed with the housing. The pressurized probe includes an electrically conductive base for electrical communication with the scalp of a user for detecting EEG signals of the user. The pressurized probe is pressurized such that the electrically conductive base is forced away from the housing and toward a user's scalp. The data collection electrode is further formed to hold a discrete amount of an electrically conductive gel therein and dispense the gel proximate the electrically conductive base to facilitate an electrical communication between the user's scalp and the electrically conductive base.

In another aspect, the pressurized probe is formed to have a reservoir therein with a dispensing hole formed at the electrically conductive base. The reservoir is used to hold the electrically conductive gel with the gel being dispensed from the reservoir through the dispensing hole.

In yet another aspect, the present invention further comprises a helmet with a plurality of electrodes are fixedly attached in predetermined patterns to the helmet. The helmet is stabilizable about a user's skull by shock absorbing pads and a chin strap.

In another aspect, the dispensing hole contains an electrically conductive captive ball dispenser that is free to rotate and limits the flow of electrically conductive gel out of the dispensing hole. Furthermore, the electrically conductive captive ball dispenser is formed of gold.

Additionally, the pressurized probe is fixedly attached to a spring that maintains the pressurization of the probe.

In yet another aspect, the data collection electrode is configured to acquire data and send it along signal wires to a Signal Processor and Transmitter.

Furthermore, the Signal Processor and Transmitter is formed to transmit data to a data repository via a radio transmitter for near-real-time data analysis.

In another aspect, the present invention further comprises a data repository configured to receive data from the Signal Processor and Transmitter and analyze the data in a manner selected from a group consisting of professional collaborative diagnosis and automated diagnosis.

As can be appreciated by one skilled in the art, the present invention also comprises a method for forming and using the system described herein.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is a cross-sectional view of an electrode according to the present invention;

FIG. 2 is a cross-sectional, rear-view of a helmet according to the present invention;

FIG. 3 is a right, side-view of the helmet according to the present invention;

FIG. 4 is a cross-sectional, left side-view of the helmet according to the present invention;

FIG. 5 is an exploded-view of components of an EEG system according to the present invention; and

FIG. 6 is a data flow diagram of a mobile in vivo EEG brain scan system according to the present invention.

### DETAILED DESCRIPTION

The present invention relates to a system for mobile electroencephalographic (EEG) data recording and, more specifically, to a system utilizing electrodes that are capable of both automatic dispensation of an electrically conductive gel and the collection of the EEG data, with a subsystem further being capable of collaborating and analyzing the acquired data. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

(1) Specific Details

As described above, the present invention relates to a system for mobile electroencephalographic (EEG) data recording. The system utilizes electrodes that are capable of both automatic dispensation of an electrically conductive gel and the collection of the EEG data. The present invention is also capable of collaborating and analyzing the acquired data. In this aspect, neural activity, in the form of field potentials, will be recorded simultaneously from multiple channels. The acquired data will be become part of a relational database management system for EEG data and will be professionally analyzed on a time-scale that approaches real-time or near-real-time.

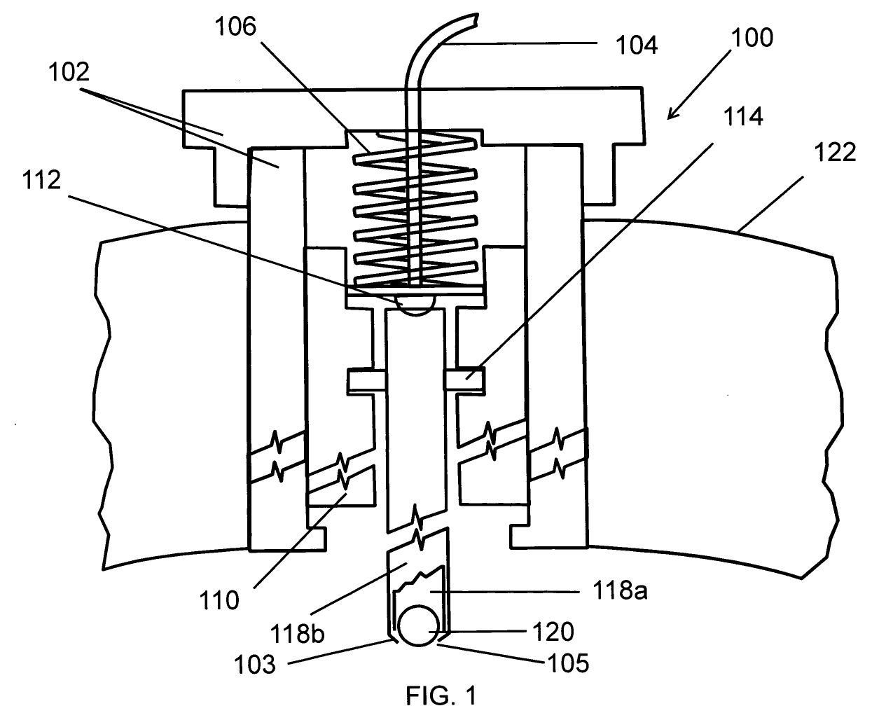

As shown in FIG. 1, the present invention includes a data collection electrode 100. The data collection electrode 100 includes a housing 102 and a pressurized, conductive probe 118b attached with the housing 102. The probe 118b includes an electrically conductive base 103 for electrical communication with the scalp of a user for detecting EEG signals of the user. The probe 118b is pressurized such that the electrically conductive base 103 is forced away from the housing 102 and toward a user's scalp. Additionally, the data collection electrode 100 is further formed to hold a discrete amount of an electrically conductive gel 118a therein and dispense the gel 118a proximate the electrically conductive base 103 to facilitate an electrical communication between the user's scalp and the electrically conductive base 103.

The probe 118b is also formed to have a reservoir therein for containing the electrically conductive gel 118a. In order to dispense the gel 118a, a dispensing hole 105 is formed at the electrically conductive base 108 that allows for fluidic communication from the reservoir to a user's scalp. An electrically conductive captive ball dispenser 120 is included that allows limited application of the electrically conductive gel 118a to the point of contact (i.e., the ball dispenser 120 and/or the base 103) of the conductive probe 118b with the user's scalp. The ball dispenser 120 is free to rotate and limits the flow of electrically conductive gel out of the dispensing hole 103. The ball dispenser 120 is formed of any suitably conductive material, a non-limiting example of which includes gold.

In a desirable aspect, the conductive probe 118b will be replaceable in order to easily replenish the reservoir of electrically conductive gel 118a. Alternatively, a top portion of the conductive probe 118b can be removed to allow a user to refill the reservoir.

To allow the probe 118b to slide within the housing 102, a slider/sleeve 110 is connected with the probe 118b and positioned within the housing 102. Both the slider/sleeve 110 and the outer electrode housing 102 are electrically insulating. Additionally, the slider/sleeve 110 and the housing 102 are made of materials that have a low coefficient of friction with one another to allow the probe 118b to slide easily within the housing 102.

A contact surface 112 is attachable (using a device such as a bayonet snap-on connector 114) with the probe 118b to transmit signals from the probe 118b to a signal wire 104. The contact surface 112 is formed in any suitable shape to facilitate an electrical connection between the probe 118b and the signal wire 104. For example, the contact surface is a hemispherical electrical contact surface with displaceable shoulder that is in direct electrical contact at its proximal face with the conductive probe 118b and at its distal face with the signal wire 104.

As mentioned above, the probe 118b is pressurized to force the probe 118b toward a user's scalp. The probe 118b is pressurized using any suitable mechanism or device, a non-limiting example of which includes a spring 106. The spring 106 is attached with the slider 110 to drive the conductive probe 118b toward the user's scalp and maintain constant pressure of the hemispherical electrical contact surface 112 (with its displaceable shoulder) with the conductive probe 118b and the electrically conductive gel. 118a

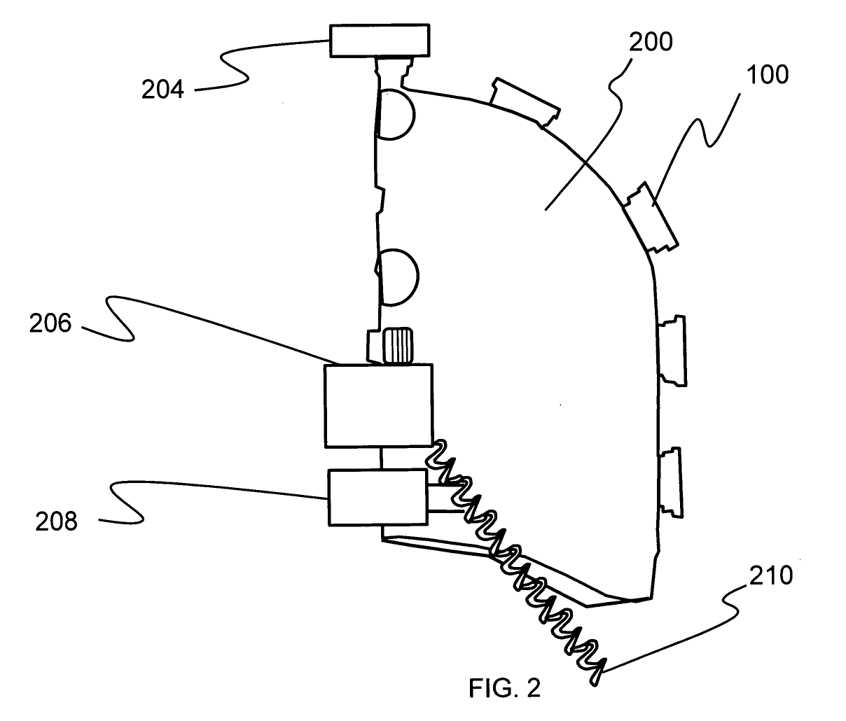

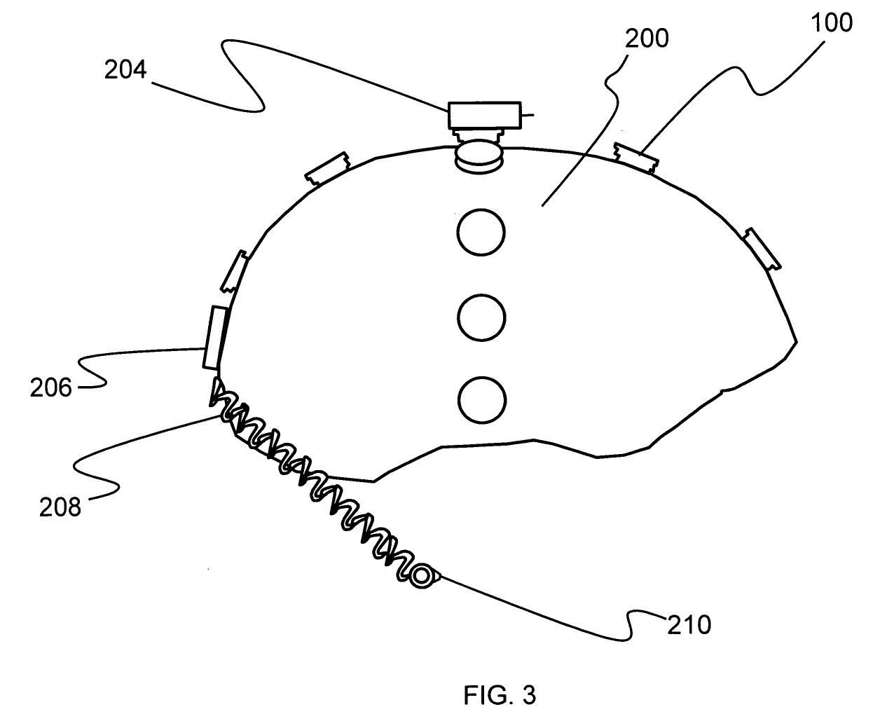

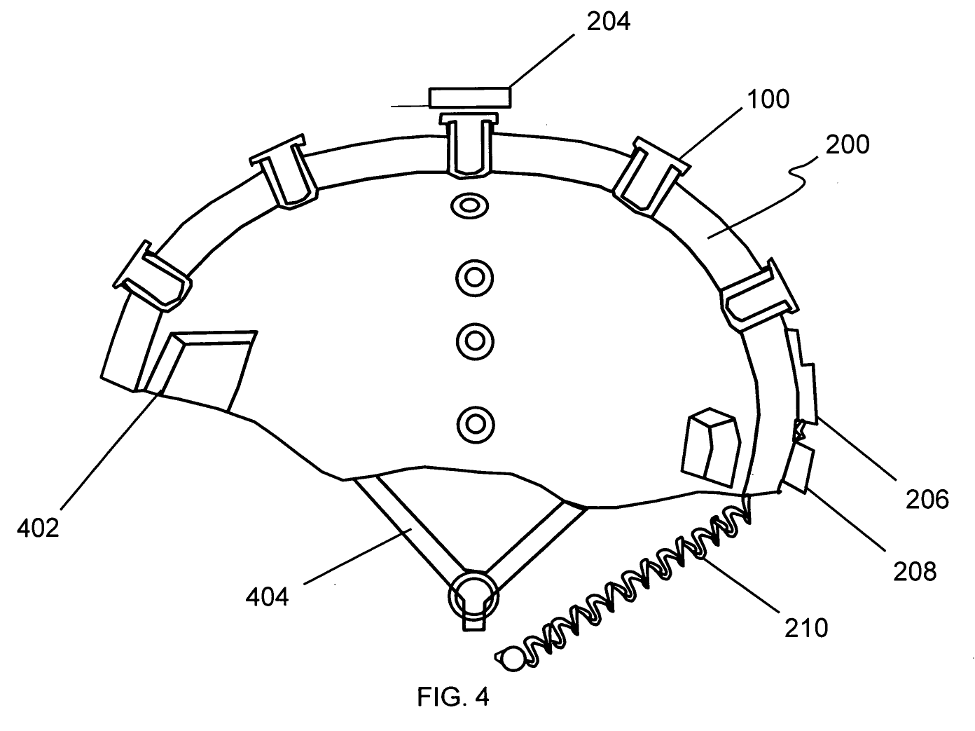

As can be appreciated by one skilled in the art, a sole electrode, in of itself, does not enable a user to capture EEG data. Thus, the present invention also includes a helmet for attaching with a user's scalp. FIGS. 2 through 4 depict various views of a helmet 200 according to the present invention. The helmet 200 is any suitable mechanism that allows a user to affix a plurality of electrodes 100 to the user's scalp, a non-limiting example of which includes a standard bicycle helmet. To facilitate in vivo usage, the helmet 200 includes shock-absorbing pads 402 and a chin-strap 404 to stabilize the helmet 200.

As described in further detail below, the present invention also allows a user to transmit the EEG data to a remote location, such as a Relational Database Management System (RDBMS). To enable such a transmission, a plurality of signal wires (shown as element 104 in FIG. 1) transfer the data from the individual electrodes 100 to a Signal Processor and Transmitter 206. Data will be transferred from the transmitter by use of any suitable transmission device, such as a patch antenna 204. Additionally, the mobile EEG system (helmet 200, electrodes 100, and requisite components) will be powered by a battery 208 or any other suitable power source. In some aspects, an EEG common ground lead 210 may be required which will serve as a reference for all recorded EEG data.

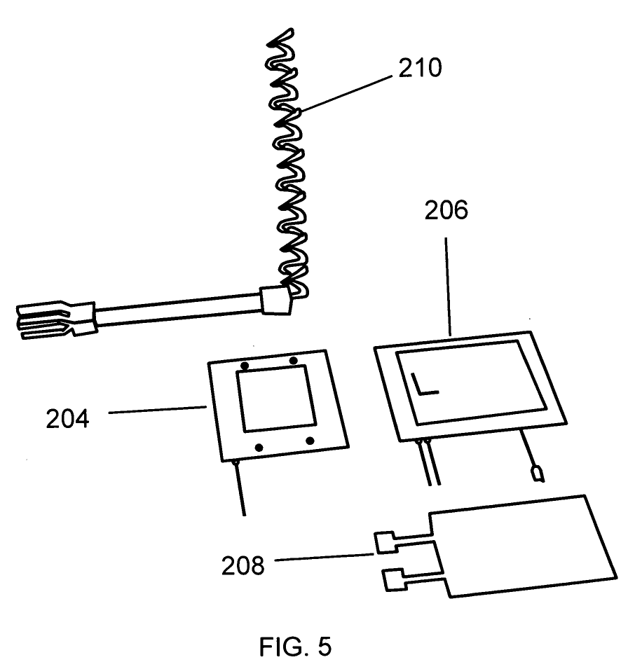

FIG. 5 further illustrates some of the important electronics utilized in the system, including the Signal Processor and Transmitter 206, the patch antenna 204, the battery 208, and the EEG common ground lead 210.

As a further description, the spring-loaded, ball-point-pen-like electrically conductive probe 118b is assembled into a small cylinder (i.e., housing 102) and is mounted in the shock-absorber lining of a helmet (described in further detail below). One or more of these cylinders will be used in the system.

In a desired aspect, these cylinders are mounted in such a way and in such numbers as to effectively replicate the typical placement and distribution of the standard, paste-on EEG probes used in medical or clinically based settings. The evoked potentials, generated from firing neuronal bundles, are picked up by these “floating” sensor probes and carried by small, insulated cables to a miniaturized multi-channel processor and radio-frequency (RF) transmitter connected to typical Patch Antennas affixed to the outside surface of the helmet. Signal sampling rates can be on the order of microseconds so as to detect multiple locations of sequentially firing neurons. These transmitted signals are received at a remote site for further processing into three-dimensional images, depicting the location of the firing neuronal bundles, and are superimposed on a translucent brain model matching the size of the subject under study. The processed signals and images are then downloaded to the RDBMS.

In another aspect, the EEG (EMF) data collected by each of the probes 118b will be passed through the small wire bundle to a Data Collection and Transmission Pack, carried in a fanny pack worn on the subject's waist. The collected data is then transmitted by the small RF Transmitter to a remote location where it is downloaded into a computerized data base for further inspection, normalization, and preparation for comparison to similar data in International Brain Data Base Systems.

The present invention also includes a diagnosis system. Misdiagnosis of neurological data based upon a variety of factors, including incomplete and misinterpreted data readouts has long been a problem in psychiatric and psychological disciplines. In addition, most diagnoses are usually arrived at through observable and thus subjective interpretation of behaviors. What is needed is a more scientific and thus objective, peer reviewed approach.

Thus, the present invention includes an EEG system that provides a means for a peer review approach to the analysis and comparison of EEG data. The analysis and comparison of these brain-wave patterns and corresponding images will be made available for study by trained medical professionals or compared to other, similar signals and images and associated diagnoses located in RDBMS's at similar international research locations.

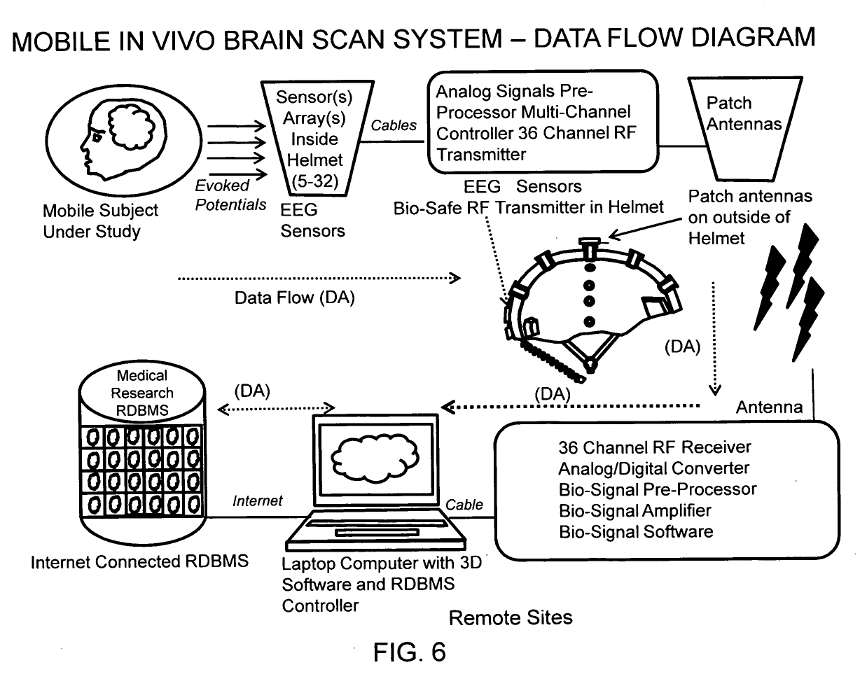

FIG. 6 illustrates such a RDMBS system according to the present invention.

The RDBMS will allow for professional cooperative collaboration in the diagnosing abnormal neural functioning that is indicative of pathology. It is a goal of the present invention to create a system for automatic classification of, or hypothesis generation for, possible diagnosis of subjects under study. The automatic classification of acquired data having traits that are consistent with certain pathologies can be achieved by directly generating (through software) a classification using markers that are decided upon via a professional collaborative effort. A drawback is that if certain aspects of EEG data that are indicative of pathology are not well described by professionals then they will not be included in the system and therefore the system would not reach its' maximum effectiveness. An alternative is to build a system that employs some form of artificial intelligence or machine learning to perform the classification. Support Vector Machines, Bayesian Networks, and in general Knowledge Based Systems are examples of possible methods that allow a system to classify acquired data as being indicative of some pathology without the need to discreetly describe all of the classification rules. The building and testing phase of a system employing artificial intelligence typically involves splitting a pre-diagnosed set of data, for example EEG data located in the RDBMS, into a learning set and a testing set.

In summary, the present invention comprises a new EEG data collection electrode that allows for mobile, in vivo EEG data collection, analysis, and diagnosis. To accomplish this, evoked potentials (EEG), generated by firing neuronal bundles in the brain, are detected by the sensors (i.e., the data collection electrodes), gently riding on the surface of the scalp. These signals are transmitted, by a small integrated multi-channel transmitter to a remote site for further computer processing into three-dimensional (3D) images which show the location (with centimeter accuracy) and the sequential timing (in microseconds) of these firing neurons. The frequency and power of the small, helmet-integrated transmitter are designed within the narrow range of non-bio-harmful parameters. The 3D images are produced using any suitable technique, such as that described by Stefan F. Filipowicz in “Identification of the internal sources with the aid of boundary element method,” as published at the International Workshop “Computational Problems of Electrical Engineering,” Zakopane, 2004.

The images produced are comparable to functional magnetic resonance imaging (fMRI), but with greater accuracy and in real time. The data is collected while the subject is mobile and functioning in a normal work or play environment. The processed images are capable of inter-active, three-dimensional manipulation and examination. The processed data can also be compared via a relational data base management system (RDBMS), through the Internet, to similar data existing in international medical and research databases, such as the Laboratory on Neural Imaging (LONI) at UCLA for comparison and validation of brain function diagnoses.

As can be appreciated by one skilled in the art, the present invention covers a wide range of brain imaging applications; such as medical triage events, physical, psychological, or other trauma.

What is claimed is:

1. An electroencephalograph (EEG) data collection system, comprising:
a data collection electrode, the data collection electrode having a housing and a pressurized probe affixed with the housing, the pressurized probe having an electrically conductive base for electrical communication with the scalp of a user for detecting EEG signals of the user, the pressurized probe being pressurized such that the electrically conductive base is forced away from the housing and toward a user's scalp, and where the data collection electrode is further formed to hold a discrete amount of an electrically conductive gel therein and dispense the gel proximate the electrically conductive base to facilitate an electrical communication between the user's scalp and the electrically conductive base.

2. An EEG data collection system as set forth in claim 1, wherein the pressurized probe is formed to have a reservoir therein with a dispensing hole formed at the electrically conductive base, where the reservoir is used to hold the electrically conductive gel with the gel being dispensed from the reservoir through the dispensing hole.

3. An EEG data collection system as set forth in claim 2, further comprising a helmet with a plurality of electrodes that are fixedly attached in predetermined patterns to the helmet that is stabilizable about a user's skull by shock absorbing pads and a chin strap.

4. An EEG data collection system as set forth in claim 3, wherein the dispensing hole contains an electrically conductive captive ball dispenser that is free to rotate and limits the flow of electrically conductive gel out of the dispensing hole.

5. An EEG data collection system as set forth in claim 4, wherein the pressurized probe is fixedly attached to a spring that maintains the pressurization of the probe.

6. An EEG data collection system as set forth in claim 5, wherein the data collection electrode is configured to acquire data and send it along signal wires to a Signal Processor and Transmitter.

7. An EEG data collection system as set forth in claim 6, wherein the Signal Processor and Transmitter is formed to transmit data to a data repository via a radio transmitter for near-real-time data analysis.

8. An EEG data collection system as set forth in claim 7, further comprising a data repository configured to receive data from the Signal Processor and Transmitter and analyze the data in a manner selected from a group consisting of professional collaborative diagnosis and automated diagnosis.

9. An EEG data collection system as set forth in claim 8, wherein the electrically conductive captive ball dispenser is formed of gold.

10. An EEG data collection system as set forth in claim 1, further comprising a helmet with a plurality of electrodes that are fixedly attached in predetermined patterns to the helmet that is stabilizable about a user's skull by shock absorbing pads and a chin strap.

11. An EEG data collection system as set forth in claim 1, wherein the dispensing hole contains an electrically conductive captive ball dispenser that is free to rotate and limits the flow of electrically conductive gel out of the dispensing hole.

12. An EEG data collection system as set forth in claim 11, wherein the electrically conductive captive ball dispenser is formed of gold.

13. An EEG data collection system as set forth in claim 1, wherein the pressurized probe is fixedly attached to a spring that maintains the pressurization of the probe.

14. An EEG data collection system as set forth in claim 1, wherein the data collection electrode is configured to acquire data and send it along signal wires to a Signal Processor and Transmitter.

15. An EEG data collection system as set forth in claim 14, wherein the Signal Processor and Transmitter is formed to transmit data to a data repository via a radio transmitter for near-real-time data analysis.

16. An EEG data collection system as set forth in claim 15, further comprising a data repository configured to receive data from the Signal Processor and Transmitter and analyze the data in a manner selected from a group consisting of professional collaborative diagnosis and automated diagnosis.

17. An electroencephalograph (EEG) data collection system, comprising:
a data collection electrode, the data collection electrode having a housing and a spring-loaded probe attached with the housing, the probe having an electrically conductive base for electrical communication with the scalp of a user for detecting EEG signals of the user, the spring-loaded probe positioned such that the electrically conductive base is forced away from the housing and toward a user's scalp, and where the data collection electrode is further formed to hold a discrete amount of an electrically conductive gel therein and dispense the gel proximate the electrically conductive base to facilitate an electrical communication between the user's scalp and the electrically conductive base.

18. An EEG data collection system as set forth in claim 17, wherein the spring-loaded probe is formed to have a reservoir therein with a dispensing hole formed at the electrically conductive base, where the reservoir is used to hold the electrically conductive gel with the gel being dispensed from the reservoir through the dispensing hole.

19. An EEG data collection system as set forth in claim 17, further comprising a helmet with a plurality of electrodes that are fixedly attached in predetermined patterns to the helmet that is stabilizable about a user's skull by shock absorbing pads and a chin strap.

20. an EEG data collection system as set forth in claim 17, wherein the dispensing hole contains a captive ball that is free to rotate and limits the flow of electrically conductive gel.

21. An EEG data collection system as set forth in claim 20, wherein the captive ball is gold.

22. An EEG data collection system as set forth in claim 17, wherein the data collection electrode is configured to acquire data and send it along signal wires to a Signal Processor and Transmitter.

23. An EEG data collection system as set forth in claim 22, wherein the Signal Processor and Transmitter is formed to transmit data to a data repository via a radio transmitter for near-real-time data analysis.

24. An electroencephalograph (EEG) data collection system, comprising:
a data collection electrode, consisting of a hollow electrically conductive tube, wherein the proximal end is in direct contact with the users scalp;
an reservoir in the data collection electrode for containing an electrically conductive gel;
an electrically conductive ball pivotally attached to the proximal end of data collection electrode, with the ball being operable to dispense the electrically conductive gel in small aliquots when the electrically conductive ball is rotated as a result of user movements,
a hemispherical electrical contact surface with displaceable shoulder attached at its proximal end to the distal end of the data collection electrode and in direct contact with the electrically conductive gel;
a spring fixedly attached to the distal end of the hemispherical electrical contact surface with displaceable shoulder;
an electrically insulating slider sheathing a portion of the medial region and the distal end of the data collection electrode;
an electrically insulating slider fixedly attached to the data collection electrode;
an electrically conductive wire that is fixedly attached to the electrical contact surface with displaceable shoulder; and
a housing that limits the degrees of freedom of the movement of the data collection electrode.
